(19) United States
(12) Patent Application Publication
(10) Pub. No.: US 2009/0214613 A1
(43) Pub. Date: Aug. 27, 2009

(54) Endothelized Artificial Matrix Comprising a Fibrin Gel, Which Is a Superproducer of Proangiogenic Factors

(75) Inventors: Jose Maria Lasso Vazquez, Madrid (ES); Paola Nava Perez, Madrid (ES)

Correspondence Address: LADAS & PARRY LLP, 26 WEST 61ST STREET, NEW YORK, NY, 10023, US

(73) Assignee: FUNDACION PARA LA INVESTIGACION BIOMEDICA DEL HOSPITAL GREGORIO MARANON, Madrid (ES)

(21) Appl. No.: 11/920,472

(22) PCT Filed: May 12, 2006
(86) PCT No.: PCT/ES2006/070059

§ 371 Date: Nov. 12, 2008

(30) Foreign Application Priority Data
May 16, 2005 (ES) P200501182

(51) Int. Cl. A61F 2/00; A61K 35/12; A61K 9/10; C12N 11/02

(52) U.S. Cl. 424/423; 424/93.21; 424/484; 435/177

(57) ABSTRACT

The invention relates to an endothelized artificial matrix comprising a fibrin gel, which is a superproducer of proantiogenic factors. The inventive matrix comprises a fibrin gel containing embedded endothelial cells which have been transfected in vitro with at least one adenoviral vector containing the sequence encoding at least one proangiogenic factor, which is inserted such that it can be overexpressed in said endothelial cells. The insertion of the aforementioned matrix between a flap and the receptor site thereof during a transplant procedure improves the survival rates of said flap, since the endothelized matrix can induce angiogenesis both in the flap and in the receptor site and, in this way, improve the vascularization of the transplanted area.

### TECHNICAL FIELD

The present invention applies to the field of artificial matrices prepared from polymeric substances present in nature, where they are seeded and make cells grow for their subsequent use in plastic and reconstructive surgery.

### BACKGROUND OF THE INVENTION

In the last few years, the development of microsurgical techniques, complemented with improved knowledge of anatomy, has been one of the great advances that have benefited Plastic and Reconstructive Surgery. Despite this, when reconstructions with flaps are made, there is a variable risk of necrosis of the same, in many cases due to vascular disturbances. The flaps are tissues in themselves (consisting of skin, muscles, bones or a combination of the same) which can be placed in anatomical areas where, due to oncological or traumatic processes, among others, a defect has been produced that requires reconstruction. Flaps have to be used when the losses of the cutaneous substance or subcutaneous tissue are not suturable or cannot heal spontaneously. The purpose of the flap is to close a loss of substance or rebuild an amputated structure. A skin flap is a piece of skin and subcutaneous cellular tissue that maintains autonomous vascularisation through the pedicle, with which it remains in contact with the deep structures. The flap pedicle is the cutaneous bridge that directly irrigates the same; sometimes it is reduced and may be represented by an artery or one or two veins. The flap is called local when the tissue that it is made from is obtained in an area near the defect that is to be repaired, and called a distant flap when the tissues are obtained from areas remote from the defect. In this last case, the flap has one artery and one vein which have to be anastomosed to another vein and artery, respectively, from the anatomical area where it is going to be located.

Thrombotic events, in both the venous and the arterial zone, and even in the micro-vessels, are the biggest problems that have to be confronted when performing a reconstruction with flaps, their appearance rate being higher in distant flaps, as they depend on microsuturing vessels of 2 mm to 5 mm in diameter. In these cases the pedicle has to be moved from its site of origin, and has to be resutured to local vessels near the area that requires reconstruction, which increases the morbidity of the process. For this reason, different methods have been sought to decrease the rate of thrombosis. There are clinical and research studies with drugs that reduce the thrombogenic potential, such as platelet antiaggregants, anticoagulants or thrombolytic agents.

Angiogenesis is the formation of new capillaries from already existing ones. It is a complex process, which may be activated in response to tissue damage. The factors involved in its stimulation are called proangiogenic factors; they play a key role in the wound healing process, decisively orchestrating the dermal neovascularisation phase. Of those, one part appears to be growth factors that are capable of stimulating the in vivo proliferation and migration of the cells that take part in the formation and stabilisation of blood capillaries. In the field of clinical practice in reconstructive surgery, growth factors also have an important role for stimulating healing in deficiency or complicated states, as happens in diabetic, oncology, and malnourished patients or those who have suffered severe traumas, in those where stress leads to a lack of all the factors that influence healing and, also, prolonged bed confinement usually increases the thrombosis risk due to their poor general state, as well as their medications. In diabetic patients in particular, neuropathy is produced, which changes the functioning of the blood vessels, or microangiopathy, which obstructs the blood capillaries, leading to a deficit in tissue perfusion which then leads to destruction of the tissue that these vessels nourish, thus the need for localised factors that accelerate the incorporation of, for example, a flap at the site where it is going to be transplanted should help to increase the vascular connections between the site and the flap and thus increase the survival rate. The role growth factors play in tissue regeneration is also important, such as, for example, when working with prefabricated flaps.

Among the growth factors that appear to be involved in the regulation of angiogenesis, fibroblast growth factors (FGF), platelet derived growth factors (PDGF), alpha-transforming growth factor (TGF-alpha) and hepatocyte growth factor (HGF), can be mentioned. Also, it has been suggested that a specific endothelial cell growth factor, vascular endothelial growth facture (VEGF) is responsible for the stimulation of growth and differentiation of endothelial cells, and certain functions of differentiated cells.

The existence of FGF (fibroblast growth factor) in the brain and in the pituitary was established by Gospodarowicz in 1974. Today, it is known that FGFs represent a group of similar proteins that act as powerful mitogens for some mesodermal and ectodermal cells.

The fibroblasts are more common in connective tissue and are adhesion cells that play an important role in aiding the healing process. The stabilisation of collagen in healing is promoted by the introduction of FGF in the site of the wounds, which appears to help in the viability of the blood vessels and promote fibroblast activity.

The angiogenic effect of FGF has also been shown in other studies. Lu et al ((Lu W W et al., Br J Plast Surg, 53: 225-229, 2000) observed that there was less ischaemia and less changes in the distribution of collagen in wounds treated with FGF, which led to a higher ability to support tautness and a higher elasticity of the tissues.

The structure and functions of acidic and basic FGF are known. Basic FGF is located in the brain, hypophysis, retina, kidneys, corpus luteum, placenta, prostate, adrenal cells and macrophages. Acid FGF is found in the brain and retina. Both stimulate endothelial cell migration and proliferation. There are studies that demonstrate the angiogenic ability and improvement in viability of flaps treated with FGF, whether the aforementioned is injected subcutaneously (Im M J et al., Ann Plast Surg, 28: 242-245, 1992) or if it is repeatedly applied using slow release pellets (Less V C et al., Br J Plast Surg 47: 349-359, 1994). In melanomas it is capable of producing angiogenesis along with other growth factors (Rofstad E K et al., Cancer Res, 60: 4719-4724, 2000) and appears that it could help in survival and the branching of myocardial arteries (Carmeliet P, Cir Res, 87: 176.178, 2000).

VEGF, for its part, was initially described as a protein secreted by tumour cells, which increased the permeability of the local cells to circulating macromolecules. It is produced by different cells in the body, among them, endothelial cells, on which it specifically acts. The direct actions of VEGF are numerous and include, among others, an increase in endothelial cell permeability. Compared to histamine, VEGF is 50,000 times more powerful as far as vascular permeability is concerned. The administration of topical VEGF produces fenestrations in the endothelium of the micro-vessels and capillaries (Roberts W G et al., J. Cell Sci, 108: 2369-2379, 1995).

During the healing process, the production of VEGF form keratinocytes is increased. This also happens in the mononuclear cells in the region where healing is taking place (Tabu P J et al, Plas Reconst Surg, 105: 1034-1041, 2000). Under physiological conditions, its production is induced by the decrease in tissue oxygen tension. The half life of VEGF under normal conditions is from 30 to 45 minutes, but under hypoxia conditions its production is extended to 6-8 hours, depending on its level of production by the tissue which is subjected to ischaemia, and the extent of tissue affected. Its production can also be increased in several diseases (Akagi K et al., Br J Can, 83: 887-891, 2000; Philipp W et al., Invest Ophtalmol Vis Sci, 41: 2514-2522, 2000).

In ischaemic areas, the endothelial cells are capable, in response to VEGF (initially liberated by inflammatory cells), of synthesising more VEGF, as well as increasing the density of the receptors for this factor in their membranes. For this reason, in an emergency situation such as ischaemia, the endothelial cells behave as producers and targets of VEGF, thus generating a chain and amplified reaction to the factor.

Several experiments have been carried out with VEGF in plastic surgery, with the aim of improving tissue perfusion. Padubiri et al (Padubiri A et al., Ann Plast Surg, 37: 604-611, 1996) injected VEGF (as recombinant protein) into the pedicle of an abdominal flap and subsequently produced an ischaemia in the same. After 7 days, the subjects treated with VEGF had a flap survival higher than those not treated. Similarly, Banbury et al (Banbury J et al., Plast Reconst Surg, 106: 1541-1546, 2000) demonstrated that it was possible to improve the perfusion of muscular flaps (cremaster muscle, in rats) subjected to ischaemia when these same rats received treatment with a VEGF perfusion in the sub-critical phase.

Studies have also been carried out to try to find the best application route for growth factors. The Kryger group (Kryger Z et al., Br J Plast Surg, 53: 234-239, 2000) designed a rat study, with the objective of comparing different application routes for VEGF in flaps. They designed six treatment groups, which were distinguished by being treated as follows: a single systemic dose of VEGF, multiple doses systemically, subcutaneously, subfascially and topically and a final control group, treated with normal saline. The best results were obtained in the group treated with multiple systemic doses of VEGF, over 72 hours. The worst result was obtained with the group treated with topically with VEGF. VEGF has also been used in prefabricated flaps. This factor appeared to accelerate the maturing of these flaps when applied in rats using polyvinyl alcohol gel (Li Q F et al., J Reconst Microsurg, 16: 45-50, 2000).

Although the results are promising, the use of growth factors such as recombinant proteins has a clear limitation, which is its short half life in vivo. Although growth factors are only needed temporarily, until the resolution of the defect, it is fundamental to obtain a therapeutic effect where the bioavailability of the factor is guaranteed during this temporary period. One of the strategies used to overcome this obstacle has been to resort to repeated doses of the factor in a fixed period (Kryger Z et al., Br J Plast Surg, 53: 234-239, 2000). A probably more efficient alternative would be to apply the growth factor not as a protein, but as a gene that is continuously expressed until the process is complete.

For this reason, the introduction of gene therapy techniques in the field of reconstructive surgery and in wound healing is of great use. Although the techniques for applying gene therapy are diverse and advancing rapidly, they mainly use viral vectors and liposome or plasmid complexes (Patterson C et al., Circulation, 102: 940-942, 2000). Adenoviruses are among the viruses being studied for use in gene therapy. They form part of a group of similar viruses, of which 47 serotypes are known. Serotypes 2 and 5 are the ones most used in gene therapy. It is a double chain DNA virus, with an icosahedral capsid. In its cycle, the viral genome resides in the nucleus, as an episomal element. They are capable of infecting a wide variety of cells.

According to Oligino (Oligino T J et al., Clin Orth, 379S: S17-30, 2000), the efficiency of the infection by adenovirus is high, compared to the lentinivirus or adeno-associated virus, although it is less than the herpes virus. Adenoviruses are not integrated in the genome of transduced cells and the duration of the transgene expression is transient, although very high. Large scale production is relatively easy. Adenovirus carriers of the VEGF gene have been used to treat patients with ischaemia of the limbs (Laitinen M et al., Hum Gene Ther, 9: 1481-86, 1998; Isner J M et al., Lancet, 348: 370-374, 1996), with a good tolerance by the patients and with no local inflammation or adverse effects. The application route was intra-arterial, although the presence of anatomical barriers, such as the lamina interna or arteriosclerosis usually reduces its efficacy. The production of growth factors with this technique generally reaches a peak at one week after treatment and the effect usually disappears at four weeks (Ylä-Hettuala S, Curr Opin Lipidol, 8: 72-76, 1997).

Another strategy for using adenovirus as vectors to provide genetic material to angiogenesis promoter cells are described in the document WO 02/36131, in which it promotes the transfection by two adenoviral vectors, each one of them containing a different form of VEGF (VEGF-B167 and VEGF-A), by injecting it in rat ears. As with the use of adenovirus VEGF carriers mentioned in the previous paragraph, the transfection is produced in vivo, therefore the angiogenesis promoter action, although it involves endothelial cells, is really non-specific. Injections of the adenoviruses were carried out in the blood vessels of the area to treat; therefore they were able to be systemically dispersed, with possible adverse effects. Although there is increased VEGF synthesis in the first hours (24-48 hours), the effect is not maintained; therefore repeated inoculations of the adenovirus are required over several days, with the subsequent discomfort to the hypothetical patient.

It would be worthwhile having an administration method available for angiogenic factors coded by virus carriers where the transfection is produced in vitro, thus permitting this transfection to be specific for endothelial cells and could avoid injecting the viruses into the blood vessels. Also, it would be advantageous if that method would enable the liberation of VEGF (or other proangiogenic factor) to be maintained over days with a single inoculation of viral vectors, making it possible for the proangiogenic factor to be available in sufficient quantities throughout the whole period of time that would be required to promote angiogenesis, but without requiring the inconvenience of repeated doses of that vector. For this reason, the matrices of the fibrin gel where the cells are made to grow are a very suitable vehicle. The fibrin provides a good base for the growth of both dermal and epidermal cells, as this protein has often been used as a support for culturing keratinocytes (Ronfard et al, Burns 17:181-184,1991).

As the fibrin does not interfere with the subsequent development of the correct dermal/epidermal binding between a wound site and the cultured keratinocytes, it has been widely used as a transport system for the aforementioned keratinocytes with the objective of repairing cutaneous lesions (Pellegrini et al., Transplantation 68: 868-879, 1999; Kaiser & Stark, Burns 20: 23-29, 1994).

Fibrin has also been used as a dermal base destined for producing large surfaces of cultured skin (Meana et al., Burns 24: 621-630, 1998). The seeded fibroblasts are able to grow inside the fibrin gels. At the same time, these fibroblasts behave as inducers of keratinocyte growth, therefore, by seeding fibroblasts and a very limited number of cultured keratinocytes over a fibrin gel, stratified confluent epithelials, very similar to normal human epithelials, are obtained in a few days (Spanish Patent ES2132027). As described in the European patent application EP 1375647, the results can be improved by using human plasma as a fundamental base for the extra-cellular matrix, which includes platelets in its composition, resuspending them in the same dermal fibroblasts to obtain an artificial dermis after coagulation of the plasma, a dermis over which keratinocytes are seeded which adhere, migrate and grow in such a way that, in a few days, a tissue consisting of two parts is obtained, an upper one, consisting of stratified epithelial cells, and a lower one, consisting of an extra-cellular matrix densely populated with fibroblasts.

With the purpose of using skin analogues similar to those described previously in transplant processes where it is attempted to replace damage skin with the aforementioned analogues, it has been proven with different strategies where there is an attempt to increase the presence of substances that they take part in angiogenesis with the aim of improving the chances of success of the artificial skin transplant. Thus, for example, the introduction of microspheres coated with fibroblast growth factor (FGF) into artificial dermis has been described (Kawai K et al., Biomaterials 21: 489-499, 2000), or inducing the production of recombinant proteins involved in angiogenesis by means of the genetic modification of keratinocytes with retroviral carrier vectors of genes of, for example, leptin hormone (WO 03/002154), VEGF (Supp et al., J Invest Dermatol 114: 5-13, 2000; Del Rio M et al., Gene Therapy 6: 1734-1741, 1999) or FGF (Erdag G et al., Molecular Therapy, 10: 76-85, 2004) or by the genetic modification of fibroblast with carrier vectors of genes that express TGF (WO 02/030443) or other angiogenic factors (WO 03/095630). However, there are no cases described where the cell modification includes or grows over a fibrin matrix that has been produced with adenovirus or cases where the cells are genetically modified and are made to grow in a fibrin matrix are endothelial cells. The nearest to this latter case would be the strategy in document U.S. Pat. No. 5,674,722, where it describes the transfection of endothelial cells so that they might synthesise some non-specific protein, using as vectors, not an adenovirus, but a retrovirus. Also, the purpose described for the transfected cells, is not to culture a fibrin matrix, but to coat a synthetic material with it that is shaped like a blood vessel. Except in this last case, in all the rest of the works mentioned the final purpose of the matrix with generated cells is to obtain a skin analogue which could be transplanted as such in patients with lesions, without describing, in any case its insertion jointly with a flap obtained directly from the anatomy of the patient to treat.

The present invention, however, proposes a different strategy, the development of vascularised bridges made from fibrin gel matrices invaded by endothelial cells, a superproducer of proangiogenic factors due to having been transfected in vitro with adenoviral vectors that contain genes that code them, with the purpose that the fibrin matrix that contains the endothelial cells act as a bridge that could be inserted between flaps of any composition (skin, muscles, bones or a combination of the same) and the anatomical part requiring reconstruction, to improve the success of the implant process. By using the aforementioned endothelialised matrix as a vascularised bridge, it speeds up the incorporation of the skin, muscle or bone flap, to the receptor site, in order to increase angiogenesis in the transplanted tissues, as well as in the receptor site itself, the latter being an advantage that is particularly important in subjects with diabetes, malnourished subjects, those who have suffered severe traumas or who have been treated with radiotherapy (for example, mastectomised women, with radiotherapy treatment, who are going to receive a reconstruction with a musculo-cutaneous free flap); subjects in whom the failure rate in flaps is usually greater, because the tissues are poorly irrigated. As explained previously, a medium like the endothelialised fibrin matrix of the invention which facilitates the formation of vascular bridges between a flap and the receptor site being of special importance in these cases. On placing the fibrin gel matrix with the endothelial cells between the flap and the radiated area, the angiogenesis produced by the gel should affect both in the same way and vascular bridges will be established between the two tissues in the first few hours after the intervention. Also, the angiogenic effect is local and more specific than that obtained by injecting adenovirus carriers of growth factor coding genes into the blood vessels, avoiding the risk of systemic dispersal, due to the transfection of the endothelial cells with the adenovirus vectors having been performed in vitro, before obtaining and implanting the vascularised bridge. The releasing of the growth factor, on the other hand, continues for days, and repeat doses are not necessary, which is more convenient for the patient. Also, unlike what might happen if the vectors used originated from a retrovirus, the use of adenovirus vectors eliminates the risk that, along with the inactivated retrovirus generated to act as carriers of the coding sequences of interest, non-inactive retrovirus genetic material is packed and, therefore, with the potential of being wholly integrated in the host cell blocking genes of interest or the blocking of which could give rise to an oncogenic process.

### DESCRIPTION OF THE INVENTION

The invention refers to an endothelialised matrix destined to be used as a vascularised bridge, composed of a fibrin gel, which supports in its interior endothelial cells capable of synthesising VEGF and/or FGF under conditions in which its synthesis would not be induced under normal conditions, due to having been transfected in vitro with adenoviral vectors that carry genes that code the aforementioned proteins. Due to those transfected genes, these endothelial cells express higher amounts of VEGF than could be produced in the normal angiogenic process that takes place in an individual receptor of a flap in a normal transplant process, therefore they have been labelled as “superproducers” of angiogenic factors in the present descriptive report.

The objective of its development is to recreate, in the laboratory, in a highly efficient way, a situation similar to that which occurs in vivo in an ischaemic area, as the aforementioned matrix invaded by endothelial cells would mimic the first stages of migration and proliferation that takes place in vivo in an ischaemic area. Once obtained, the final purpose of the endothelialised matrix is its insertion as an intermediate element between a flap used in the reconstruction process and the receptor site that receives it, such that, after the transplant, the matrix inserted like a vascular bridge acts on the individual receptor as a strong inducer of angiogenesis. To increase the performance of the system, the endothelial cells, before being seeded in the matrix, are converted into superproducers of proangiogenesis factors (VEGF and/or FGF) using a gene transference protocol with adenoviral vectors that carry its coding sequence and control elements that enables it to be expressed in endothelial cells. The matrix used, a fibrin gel, acts as an optimal support for cell proliferation and migration as well as for the production of the factors.

This system, which combines the introduction of endothelial cells into the matrix with the sustained in situ production (but for a limited time) of proangiogenic factors, represents a clear advantage over the topical or systemic administration of growth factors such as recombinant proteins or by injecting adenoviral vectors that contain genes that code the aforementioned recombinant proteins with the aim of obtaining an in vivo transfection process.

Another objective of the present invention is a method for the production of the aforementioned superproducer endothelialised fibrin matrix of at least one proangiogenic factor due to its endothelial cells having been partly or completely transfected in vitro with one or more adenoviral vectors which have at least one gene corresponding to a proangiogenic factor in their sequence, which consists of the following steps:
a) to obtain individualised endothelial cells after having been isolated from a mammal and cultured in vitro;
b) to transfect in vitro a part or all the aforementioned endothelial cells with one or more different adenoviral vectors which contain in their sequence at least one gene corresponding to a proangiogenic factor inserted in such a way that the gene is able to be expressed in endothelial cells;
c) to mix the medium that contains the endothelial cells transfected in the previous step with a solution that contains fibrinogen and to stimulate the gelling of the fibrinogen to form fibrin;
d) to allow the mixture from the previous step to stand in a suitable receptacle so that the formation of the fibrin gel matrix is produced in which the endothelial cells transfected with adenoviral vectors have been left to soak.

Similarly, it is an objective of the invention to use the superproducer of proangiogenic factors endothelialised matrix as a vascularised bridge to insert between a flap and a receptor site of the same, to improve the survival of the said flap.

In a preferred realisation of the invention, the superproducer of proangiogenic factors endothelialised fibrin matrix will be designed with the aim that the receiver individual for whom it is foreseen would be human.

In a realisation of the invention, the individual from whom the endothelial cells as well as the fibrinogen from which the fibrin originates is the same as that foreseen as the receiver individual of the endothelialised matrix, the matrix being completely autologous.

In another realisation of the invention, the matrix is not autologous, individuals different from the one foreseen as the receiver of the matrix being possible as donors of endothelial cells and/or fibrinogen. The donating and receiving individuals could even belong to different species.

### SHORT DESCRIPTION OF THE FIGURES

FIG. 1 is a schematic representation of a dissected flap. (1): flap; (2): avascular site; (3): artery; (4): vein.

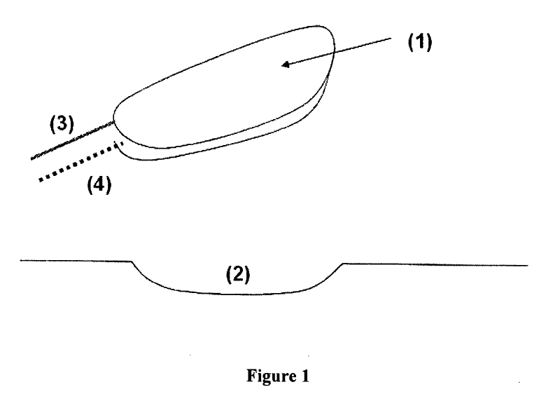

FIG. 2 is a schematic representation of the way in which the flap (1) and the endothelialised matrix (5) that will act as a vascularised bridge will be placed in relation to the receiver site (2). An artery (3) and a vein (4) are again shown in the flap.

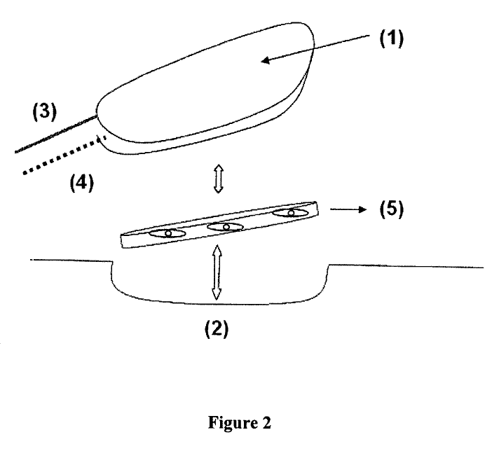

FIG. 3 is a photograph showing the flap design, on the dorsal side of the ear of a rabbit and with the axis centred in the intermediate caudal vessels. The endothelialised matrix being distributed over the cartilage situated below the flap.

FIG. 4 shows immunohistochemical stains of CD32 in treated subjects (part a) and control subjects (b) at 500 magnification.

FIG. 5 shows a photograph of vessels of a receiver individual of a flap, treated with endothelialised fibrin gel matrix, with a positive reaction as regards VEGF in its endothelial cells.

### DETAILED DESCRIPTION OF THE INVENTION

The present invention, therefore, provides a fibrin matrix that has, inside it, endothelial cells transfected with adenoviral vectors and for this reason superproducers of VEGF and/or FGF, a matrix that is prepared with the purpose of being used as a connecting vascularised bridge between the receiver site and a flap.

The endothelial cells have been extracted previously from peripheral veins of an individual of the same species, which can be the actual subject to treat, and are cultured and genetically modified to finally be absorbed into a matrix of fibrin gel obtained from plasma, usually also from the actual patient. The genetic modification of the endothelial cells is produced with adenovirus carriers of the VEGF and/or FGF genes, in such a way that, in vivo, they behave as bioreactors of the aforementioned factors for a limited time only, while the aforementioned cells transfected with the adenovirus derived vector survive in the fibrin gel. This endothelialised vector and superproducer of proangiogenic factors has the purpose of acting as a vascularised bridge to induce the development of a functional vascular plexus between the flap and the receptor site, with the aim of accelerating the adaptation between both. The fibrin gel matrix provides a suitable environment so that growth factors may generate and accumulate in it.

The advantages of using a bridge vascularised by an endothelialised matrix composed of a matrix of fibrin gel in which endothelial cells that are superproducers of proangiogenic cells due to having been transfected in vitro with adenoviral vectors that contain genes that code the aforementioned proangiogenic facts are absorbed, are as follows:
The fibrin gel matrix allows the growth and migration of endothelial cells within it. Inside this matrix, genetically modified endothelial cells are capable, in vitro, of secreting proangiogenic growth factors (VEGF and FGF) and being organised by forming micro-capillaries before being transplanted.
After transplanting this vascularised bridge, placed between the receptor site and the flap, the growth factors produced by the genetically modified cells are also capable of inducing the proliferation and migration of new vessels in the receptor site as well as in the flap itself. The endothelial cells of the vascularised matrix should act as a bridge between both, many of them ending up by being included as part of the vascular stroma which will bind the transplanted tissue to the receptor site, such that, overall, increases the possibilities of success in the reconnection of the flap.
The increase in angiogenesis in the transplanted tissue helps to accelerate the incorporation of a flap of skin, muscle, or bone tissue or a combination of these tissues to the receptor site.
The use of this endothelialised tissue as a vascularised bridge is of great use not only for reducing thrombotic events that could threaten the survival of the flaps, but also for the treatment of flaps that may have to be used to reconstruct areas treated with radiotherapy, flaps in diabetic patients or smokers (who usually have micro- and/or macrovascular problems), and also to prepare prefabricated flaps.
The fact that the genetic modification of the endothelialised cells is produced by an in vitro transfection, before the matrix transplant, ensures that the incorporation of the genetic material is produced specifically in the cells desired and has a clear advantage over the in vivo injection of viral vectors, as it decreases the risk of systemic dispersal, allows a continued release effect of growth factors for days and does not require repeat doses.
The use of adenovirus as carrier vectors is also an advantage compared to the use of a retrovirus, since it avoids the risk of a possible packing together with inactivated vectors of genetic material with oncogenic potential and their possible integration into the host cell in a stable form and blocking other genes.
The possibility that all the components of the endothelialised matrix may be of autologous origin means that, in those cases, the insertion of the matrix as an endothelialised bridge and the corresponding flap may be performed without the need for immunosuppression of the treated subjects.
On the other hand, the flexibility within the method for obtaining the endothelialised matrix means that the serum used to form the fibrin gel as well the endothelialised cells that are transfected and soaked into the matrix could come from a different individual from that who is going to have the flap inserted. Even the species from which the fibrinogen of the fibrin matrix originates can be different. This opens the possibility that matrices may be prepared in advance when they are required urgently, although in these cases it would be advisable to give immunosuppression to the subjects in whom the endothelialised matrix is implanted as a vascularised bridge.

To achieve this, endothelial cells have to be produced, transfected in vitro with an adenoviral vector that enables the expression of VEGF and/or FGF. The aforementioned endothelial cells are extracted from the peripheral nervous system, preferably from the saphenous vein, of the donor. If the endothelialised matrix needs to be completely autologous, the donor will have to be the same individual who needs the flap. The extracted vessels are sent in a transport flask with DMEM and an antiseptic solution for their culture and preparation, using, for example, the method described by Del Rio et al, Br J Pharmacol, 120:1360-1366, 1997. Following this method, the endothelial cells are cultured in a suitable medium, like, perhaps, the modified Dulbecco medium: Hams F12 (1:1) which contains 10% FCS supplemented with glutamax 1, 100 IU/ml penicillin G, 100 μg/ml streptomycin and 0.25 μg/ml amphotericin B.

The in vitro genetic transfer is carried out with confluent cultures by incubating them with adenovirus vectors that carry the genes that code the growth factors (VEGF, FGF) in a serum poor medium. After two washes with PBS, the cells are incubated in a suitable culture medium, like, perhaps, Dulbecco: Ham's F12 (1:1), for a suitable time which, in the case of humans, would be approximately 24 hours. Then, to obtain the individual cells that will form part of the endothelialised matrix, they are treated with trypsin/EDTA.

To prepare the endothelialised matrix, the method described previously in the international patent application WO 02/072800 can be used. In it, the plasma is separated, with platelets and fibroblasts, which gel by using Ca²⁺. Unlike that in the aforementioned document, in the present invention the fibroblasts are not resuspended in the fibrin matrix, nor are the keratinocytes seeded in it, but the type of cells that are resuspended in the matrix are endothelial cells which are obtained as described in the previous paragraph, which are modified genetically. In this way, the matrix of the fibrin gel of the present invention not only acts as a cellular support, but also serves as a vehicle of therapeutic factors produced by the cells.

The fibrin matrix can also be obtained from its blood precursor, fibrinogen, from plasma cryoprecipitates. The cryoprecipitates are obtained in accordance with the standards of the American Association of Blood Banks (Walker R H (ed) Technical Manual, American Association of Blood Banks, Bethesda, Md.; 1993; pp. 728-730).

The individual from whom the plasma is extracted may or may not be that in whom the flap is going to be inserted and, in this flap, the endothelialised artificial matrix consisting of a superproducer of proangiogenic factors. It is preferred that the individual is the same person if it is desired to avoid the need of subjecting the receiver of the flap to immunosuppressive treatment. In cases where this is not a fundamental factor, the plasma may come from not only a different individual, but also from a different species. Thus, the fibrinogen source can be, for example, porcine plasma cryoprecipitates.

In any of the cases, to produce the fibrin gel, DMEM which contains 1% FCS and the already transfected endothelial cells are added to the fibrinogen solution. Subsequently, gelification is induced by the addition of CaCl² and thrombin. Finally, the mixture is poured over a culture plate or other suitable receptacle and is left to solidify at a suitable temperature, which will be 37° C. in the case of fibrin gels originating from humans. The gel formed is covered with a suitable culture medium, (for example, Dulbecco: Ham's F 12 (1:1) and 24-48 hours afterwards it is transplanted over the receptor site of the flap, so that it acts as a vascularised bridge between both. Once the flap is positioned over the endothelialised matrix, the edges of the same are sutured to the site itself, in such a way that the endothelialised matrix remains homogeneously distributed between both.

As is described in more detail below in the corresponding example, surgical experiments performed on animals, specifically rabbits, verified the validity of the method and its usefulness in increasing the survival of inserted flaps. Also, after performing a statistical analysis, the results showed that the capillary density and the VEGF expression were significantly better in the treated subjects.

### EXAMPLE

### Preparation of the Endothelial Cells

The endothelial cells used to be lodged in the fibrin matrix were endothelial cells from the aorta of New Zealand albino rabbits. These same cells were cultivated after extracting them from the aortic artery of these rabbits under sterile conditions and in a culture medium (5% DMEM, with an antibiotic and anti-fungicide). The cells were cultured in a medium modified by Dulbecco: Ham's F12 (1:1), which contains 10% FCS, supplemented with glutamax 1, 100 IU/ml penicillin G, 100 μg/ml of streptomycin and 0.25 μg/ml amphotericin B.

In the study, cells were used that had been subjected to three steps at the most during their culture. The in vitro genetic transfer was performed in confluent cultures, by incubation for 3 hours at 37° C. with a Group C adenoviral vector, which included a gene of VEGF A 165, capable of being expressed in endothelial cells in a serum-poor medium. After two washes with PBS, the cells were incubated in a growth medium for 24 hours and later treated with trypsin/EDTA, to obtain individual cells.

### Preparation of the Endothelialised Fibrin Matrices

The fibrin gels containing the transfected cells were prepared following the protocol for fibroblast fibrin gels described in the international patent application WO 02/072800, with modifications. Firstly, fibrinogen from porcine plasma cryoprecipitates was used as a resource for obtaining the fibrin. The cryoprecipitates were obtained in accordance with the standards of the American Association of Blood Banks. To produce the fibrin gel, 3 ml of the fibrinogen solution were added to 12 ml of DMEM in 10% FCS, with 5×10⁵ transfected endothelial cells. Then 1 ml of CaCl₂ (0.025 mM, Sigma) was added along with 11 IU bovine thrombin (Sigma). Finally, the mixture was poured into a 75 cm² culture receptacle and left to solidify at 37° C. The gel is covered with a culture medium to be used after 24 hours, being kept in a refrigerator at 4° C. during this time interval.

### Surgical Procedure

The animals in which the flaps were inserted were also New Zealand albino rabbits, although those individual from whom the endothelial cells had been obtained were not used. For this reason, as well as due to the use of porcine plasma as a fibrin source, immunosuppression had to be provoked in the subjects treated, which in this case was carried out with Sandimmune® (Novartis Pharmaceuticals), using an intraperitoneal dose of 25 mg/kg the day before the operation. This dose was repeated daily in all the subjects of the study, until they were sacrificed.

As regards obtaining the flaps, axial flaps were designed from the dorsal region of the ear of each rabbit, which is based in the intermediate branch of the artery and caudal auricular vein. The proximal edge of each flap is situated 4 cm distal to the junction of the median caudal auricular vein with the corresponding caudal vein of the ear. Under aseptic and antiseptic conditions, the edges were infiltrated with local anaesthetic and an incision was made on the edges with a scalpel, trying not to section the vascular pedicle. On the distal edge of the flap, after isolating the central vessels, electro-coagulation is performed on the same. Using blunt-end scissors, the flap is separated from the cartilaginous tissue, observing the central vessel insertions to the perichondrium. Later, with the aid of a scalpel, the whole flap was removed in a proximal direction. With a scalpel incision, the vessels in the proximal area of the axial flap are accessed, up to the junction of the median caudal auricular vein with the corresponding caudal vein. At this time the caudal vein is coagulated, to avoid any interference of this vessel with the axial vessel of the flap. The perichondrium was then removed from the cartilage in the exposed area. With the aid of magnified glasses and microsurgery tools, the blood vessels are isolated and the nerve fillets that are attached to the central vessels were sectioned, because these contain small accompanying vessels that could nurture the actual flap themselves.

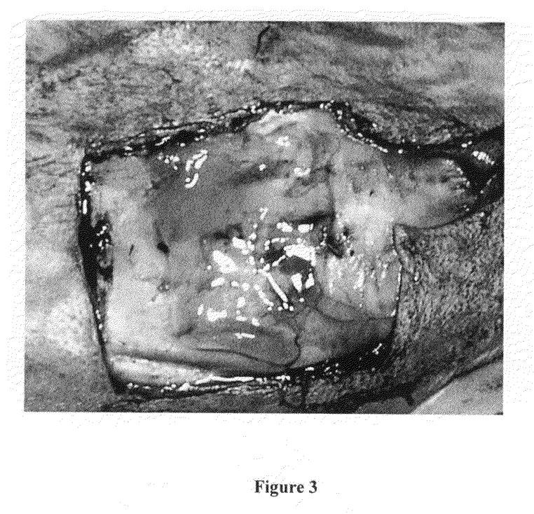

The endothelialised fibrin matrix is handled in a laminar flow hood, to remove it from the glass receptacle that contains it, with the aid of a spatula. The capsule is then covered with sterile paper and is transported to the operating theatre for its implantation over the cartilage with its perichondrium removed. It was ensured that the distribution of the matrix was as homogeneous as possible. The flap was positioned over this. Using a silk suture (4/0), the edges of the same and the incision made to expose the vessels were sutured. An example of the final arrangement is shown in FIG. 3.

Once operated on, the animals were returned to their cages.

The final surgical act consisted in sectioning the vessels of the axial flap. For this, the same anaesthetic procedure was performed again, although in this case, the infiltration with 2% lidocaine was made at ½ cm from the proximal edge of the flap. An incision was made with a scalpel over the surgical scar itself and with the aid of microsurgery clamp the arterial and venous flow was cut off. Then, the vessels were sectioned and ligated. In group I (control) and II (treated with the VEGF producer matrix), this procedure was carried out at 5 days from the date of removing the flap. In group III (control) and IV (treated with the VEGF producer matrix), the sectioning was performed 48 hours after carrying out the initial surgery.

### Treatment of the Operated Animals

Once the animals were returned to their cages after the first intervention, a daily assessment was made of each subject, making a note of details of the colour of the flaps and its consistency to touch.

Four days after performing the sectioning of the vessels, photographs were taken of them and, at 6 days, the animals were killed with an overdose of intravenous sodium pentothal.

At this time the flaps were extracted and placed in receptacles with 10% buffered formol. At 48 hours, three portions from the proximal, medium and distal areas, respectively of each flap were selected, in such a way that the central vessels were in the centre of the histology section, and were embedded in paraffin.

### Macroscopic and Microscopic Evaluation

The flap surface that was viable was assessed by planimetry; the values obtained being expressed as percentages. The aforementioned planimetry was performed the day before the animal was sacrificed.

Sections of 3-4 μm were made from the paraffin block, which were mounted on silanised slides, with a positive surface charge and a capillary gap of 75 μm (ProbeOn™ Plus Slides. Catalogue No. 15-188-52. Fischer Biotech® and ChemMate™ Capillary Gap Plus Slides. Code S2024: DAKO A/S Biotek Solutions). After paraffin removal and hydration, the slices were washed in Tris saline (TBS) (0.05 M Tris-HCl; 0.5 M NaCl; pH 7.36). Endogenous peroxidase activity was blocked using 3% hydrogen peroxide in methanol for 30 minutes.

With the intention of exposing the highest possible number of epitopes, by unfolding the proteins by denaturation, the sections were subjected to a pre-treatment with heat in a microwave at 750 watts, submerged in a citrate buffer (0.01 m citric acid, pH 7, for four periods of five minutes, then leaving them to cool to room temperature.

The non-specific background staining block was done by using non-immune normal goat serum (Code NGS-1, University of Navarre, Pamplona), diluted 1:20, for 30 minutes at room temperature.

The slices were incubated with CD31 antibodies (CD-31 mouse monoclonal antibody. Code M 0823. DAKO), with a 1/100 dilution, and anti-VEGF (VEGF (C-1): sc-7269. Santa Cruz Biotechnology, Inc.). The CD31 protein is specific for endothelial cell membranes; therefore, the stains that can detect those sites to which the CD-31 antibodies will bind enable the blood vessel walls to be visualised and, therefore, their presence.

After washing in TBS (0.05 M Tris-HCl; 0.5 M NaCl; pH 7.36), it is incubated for 30 minutes at room temperature, with the EnVision™ product, peroxidase (DAKO EnVision™. Code No. K4003 anti-rabbit; Code No. K4001 anti-mouse) pre-diluted.

After the final wash in TBS (0.05 M Tris-HCl; 0.5 M NaCl; pH 7.36), the product of the peroxidase reaction is visualised using a commercially prepared 3,3′-diaminobenzidine (DAB) solution in a chromogenic solution, with a imidazole-HCl buffer at pH 7.5 and hydrogen peroxide (DAKOc Liquid DAB+Large Volume Substrate-Chromogen Solution Code No. K3468), incubating it for five to thirty minutes, at room temperature and pre-diluted.

A pathologist is responsible for carrying out the evaluation of the histological preparations which have been stained with haematoxylin-eosin and immunohistochemical stains (CD31 and VEGF).

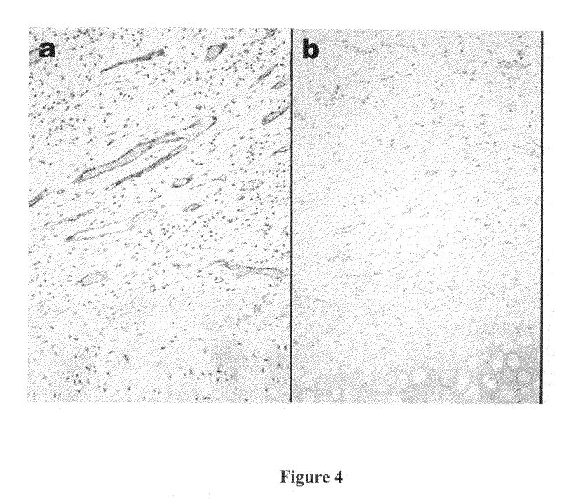

In the CD31 stains, the vessels are counted, at ×500 magnification, in 6 different fields and the mean value of the same is expressed. These areas where there had been an inflammatory focus were ignored and the areas where there had not been any subcutaneous tissue distortion between the skin appendages themselves and the cartilage were evaluated. FIG. 4 shows examples of these CD31 immunohistochemical stains in treated subjects (a) and control subjects (b). The count results are shown later in Table 1, which corroborate that there is a greater formation of blood vessels in the treated subjects with the matrix of the invention compared to the subjects in the control groups. The associated statistical parameters are shown in Tables 2 and 3.

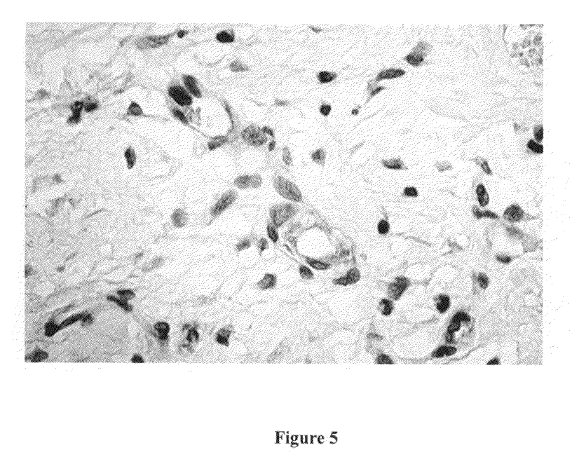

A regards VEGF, those vessels in which the cytoplasm of the endothelial cells was stained were considered positive. The count was made giving a numerical value to all the cells that were stained with antibody in each preparation. An example of vessels stained with a positive reaction for VEGF in endothelial cells in an individual treated with the endothelialised fibrin gel of the invention is shown in FIG. 5, where the results of the stained cells demonstrate the presence of VEGF and, therefore, that it being synthesised effectively. The count results of the endothelial cells stained are shown later in Table 1. The associated statistical parameters are shown in Tables 2 and 3.

### Results

Table 1 presented below shows the data of the means and standard deviations corresponding to the survival data, CD31 stains and VEGF stains.

TABLE 1
Means and standard deviations corresponding to
the survival data, CD31 stains and VEGF stains.

| | | MEAN/STANDARD DEVIATION | | |
|---|---|---|---|---|
| | | | CD31 | VEGF |
| | | SURVIVAL | Vessels/field* | Endothelial |
| | | (%) | 500 | cells/section |
| Section | Group 1 | 51.25/45.88 | 5.56/3.18 | 0.87/1.12 |
| at 5 days | (control) | | | |
| | Group II | 95.62/4.95 | 13.20/4.54 | 5.62/3.73 |
| | (treatment) | | | |
| Section at | Group III | 2.50/7.07 | 2.34/4.56 | 0.37/1.06 |
| 48 Hours | (Control) | | | |
| | Group IV | 55.62/38.95 | 5.56/3.18 | 3.06/2.95 |
| | (treatment) | | | |

Below, in Tables 2 and 3, the data corresponding to the statistical parameters associated with the previous results are shown, specifically those relative to the Kruskal Wallis (Table 2) and the Whitney-Mann U (Table 3) analysis.

The analysis of variance (ANOVA) is a data analysis technique to examine the significance of the factors (independent variables) in a multifactorial model. The single-factorial model can be obtained from a generalisation of a two-sample test. That is, a test of two samples is that part from the hypothesis that the means of the populations are equal. The ANOVA test will assess the hypothesis that defends that the means of “x” populations are equal.

The Kruskal Wallis test may be used like ANOVA. It is a non-parametric test which is used when the conditions to use the ANOVA test cannot be applied, that is, to contrast the hypothesis that a number of different sized samples originate from the same population. Thus, the Kruskal-Wallis test is a non-parametric method to evaluate the hypothesis that several populations have the same continuous distribution versus the alternative that results tend to be different in one or more populations.

TABLE 2
Kruskal-Wallis parameters

| Kruskal Wallis | SURVIVAL | CD31 | VEGF |
|---|---|---|---|
| X² | 15.50 | 17.11 | 16.38 |
| P | 0.001 | 0.001 | 0.001 |

As for Table 3, the Mann-Whitney U test is a non-parametric statistical test that is used when the sample is small or the distribution of the data in the population is free (data not originating from normal populations and with similar variances). This test compares whether two samples of two sub-populations have the same distribution.

The observations of both groups are combined and classified according to the average range assigned in case ties are produced. If the position of the populations is identical, the ranges should be randomly mixed in both samples.

TABLE 3
Mann-Whitney U analysis

| | | P | | |
|---|---|---|---|---|
| U Mann Whitney | Pairs | SURVIVAL | CD31 | VEGF |
| Section in 5 days | | 0.08 | 0.00 | 0.00 |
| (treatment versus control) | | | | |
| Section in 2 days | | 0.00 | 0.05 | 0.02 |
| (treatment versus control) | | | | |

Therefore, according to these data, a survival of the flaps of around 50% was found in treated subjects despite dispensing with the pedicle at 48 hours from being intervened (Group IV), a fact which makes it easier for the flap to necrose, as the blood flow of the flap depends on the pedicle. The survival increased to 95% if the section of the pedicle was performed at 5 days (protocol A). In the non-treated subjects, the survival did not reach 3% after sectioning the pedicle at 48 hours.

The results showing the capillary density and the VEGF expression were also significantly better in the treated subjects.

### SUMMARY

Endothelialised artificial matrix consisting of a fibrin gel which is a superproducer of proangiogenic factors. The matrix consists of a fibrin gel in which is embedded endothelial cells that have been transfected with at least one adenoviral vector which contains the coding sequence of at least one proangiogenic factor inserted in such a way that it can be over-expressed in these endothelial cells. The insertion of the aforementioned matrix between a flap and its receptor site in a transplant process improves the survival rates of said flap, as the endothelialised matrix is able to induce angiogenesis both in the flap and the receptor site thus improving the vascularisation of the transplanted area.

1. An artificial matrix of an endothelialised fibrin gel which contains endothelial cells embedded in its interior that, in part or in its entirety, has been transfected in vitro with one or more adenoviral vectors which has in its sequence at least one gene corresponding to a proangiogenic factor capable of overexpression in the aforementioned transfected endothelial cells.

2. An artificial matrix of a superproducer endothelialised fibrin gel of at least one proangiogenic factor according to claim 1, in which the endothelial cells originate from the venous system of a mammal.

3. An artificial matrix of a superproducer endothelialised fibrin gel of at least one proangiogenic factor according to claim 2, in which the endothelial cells specifically originate from the saphenous vein.

4. An artificial matrix of a superproducer endothelialised fibrin gel of at least one proangiogenic factor according to claim 1, in which the endothelial cells originate from the arterial system of a mammal.

5. An artificial matrix of a superproducer endothelialised fibrin gel of at least one proangiogenic factor according to claim 4, in which the endothelial cells specifically originate from the aortic artery.

6. An artificial matrix of a superproducer endothelialised fibrin gel of at least one proangiogenic factor according to claim 1, in which the fibrin gel has been formed from fibrinogen present in blood plasma of a mammal.

7. An artificial matrix of a superproducer endothelialised fibrin gel of at least one proangiogenic factor according to claim 1, in which the fibrin gel has been formed from fibrinogen of plasma cryoprecipitates of a mammal.

8. An artificial matrix of a superproducer endothelialised fibrin gel of at least one proangiogenic factor according to claim 1, in which at least one adenovirus used to transfect the endothelial cells contains in its nucleotide sequence the coding sequence of the growth factor VEGF capable of being overexpressed in the aforementioned endothelial cells.

9. An artificial matrix of a superproducer endothelialised fibrin gel of at least one proangiogenic factor according to claim 1, in which at least one adenovirus used to transfect the endothelial cells contains in its nucleotide sequence the coding sequence of the growth factor FGF capable of being overexpressed in the aforementioned endothelial cells.

10. A method for obtaining an artificial matrix of a superproducer endothelialised fibrin gel of at least one proangiogenic factor, which comprises the following steps:
a) obtaining individualised endothelial cells after having been isolated from a mammal and cultured in vitro;
b) to partly or completely transfecting in vitro aforementioned endothelial cells with one or more different adenovirus vectors which contain their sequence at least one gene corresponding to a proangiogenic factor capable of being overexpressed in the aforementioned endothelial cells;
c) mixing the medium that contains the endothelial cells transfected in the previous step with a solution that contains fibrinogen and to stimulate the gelling of the fibrinogen to form fibrin,
d) allowing the mixture from the previous step to stand in a suitable receptacle so that the formation of the fibrin gel matrix is produced in which the endothelial cells transfected with adenoviral vectors have been left embedded.

11. A method for obtaining an artificial matrix of a superproducer endothelialised fibrin gel of at least one proangiogenic factor according to claim 10, in which the stimulation of the gelling of the fibrinogen for the formation of fibrin is by the addition of CaCl₂ and thrombin.

12. A method for obtaining an artificial matrix of a superproducer endothelialised fibrin gel of at least one proangiogenic factor according to claim 10, in which the endothelial cells originate from the venous system of a mammal.

13. A method for obtaining a matrix of a superproducer endothelialised fibrin gel of at least one proangiogenic factor according to claim 12, in which the endothelial cells originate specifically from the saphenous vein.

14. A method for obtaining a matrix of a superproducer endothelialised fibrin gel of at least one proangiogenic factor according to claim 10, endothelial cells originate from the arterial system of a mammal.

15. A method for obtaining a matrix of a superproducer endothelialised fibrin gel of at least one proangiogenic factor according to claim 14, in which the endothelial cells specifically originate from the aortic artery.

16. A method for obtaining a matrix of a superproducer endothelialised fibrin gel of at least one proangiogenic factor according to claim 10, in which the fibrin gel has been formed from fibrinogen present in blood plasma of a mammal.

17. A method for obtaining a matrix of a superproducer endothelialised fibrin gel of at least one proangiogenic factor according to claim 10, in which the fibrin gel has been formed from fibrinogen of plasma cryoprecipitates of a mammal.

18. A method for obtaining a matrix of a superproducer endothelialised fibrin gel of at least one proangiogenic factor according to claim 10, in which at least one adenovirus used to transfect the endothelial cells contains in its nucleotide sequence the coding sequence of the growth factor VEGF capable of being overexpressed in the aforementioned endothelial cells.

19. A method for obtaining a matrix of a superproducer endothelialised fibrin gel of at least one proangiogenic factor according to claim 10, in which at least one adenovirus used to transfect the endothelial cells contains in its nucleotide sequence the coding sequence of the growth factor FGF capable of being overexpressed in the aforementioned endothelial cells.

20. In an operation to implant a tissue flap in a mammal, a method of producing a vascularized bridge between said flap and tissue of a recipient thereof which comprises inserting a matrix of a superproducer endothelialised fibrin gel of at least one proangiogenic factor between said flap and a receptor site therefor wherein said matrix is an artificial matrix of an endothelialised fibrin gel which contains endothelial cells embedded in its interior that, in part or in its entirety, has been transfected in vitro with one or more adenoviral vectors which has in its sequence at least one gene corresponding to a proangiogenic factor capable of overexpression in the aforementioned transfected endothelial cells.

21. A method according to claim 20, in which endothelial cells present in the fibrin gel matrix originate from a different individual from the one who receives the flap.

22. A method according to claim 20, in which endothelial cells present in the fibrin gel matrix originate from the same individual who receives the flap.

23. A method according to claim 20, in which endothelial cells present in the fibrin gel matrix originate is a non-human mammal.

24. A method according to claim 20, in which the recipient of the flap is human.

25. A method according to claim 20, in which the recipient of the flap is a non-human mammal.

26. A method according to claim 20, in which the fibrin gel of the artificial matrix has been formed from fibrinogen present in the blood plasma of a different individual from the one who receives the flap.

27. A method according to claim 20, in which the fibrin gel has been formed from fibrinogen present in the blood plasma of the same individual that receives the flap.

28. A method according to claim 26, in which the individual from whom the blood plasma originates and from which the fibrinogen that forms the fibrin gel of the artificial matrix is obtained is a non-human mammal.

29. A method according to claim 26, in which the recipient of the flap is human.

30. A method according to claim 26, in which the recipient of the flap is a non-human mammal.

31. A method according to claim 20, in which the fibrin gel of the artificial matrix has been formed from fibrinogen present in the blood plasma cryoprecipitates of a different individual from the one who receives the flap.

32. A method according to claim 20, in which the fibrin gel of the artificial matrix has been formed from fibrinogen present in the blood plasma cryoprecipitates of the same individual who receives the flap.

33. A method according to claim 31, in which the individual from whom the blood plasma cryoprecipitate originates and from which the fibrinogen that forms the fibrin gel of the artificial matrix is obtained is a non-human mammal.

34. A method according to claim 31, in which the recipient of the flap is human.

35. A method according to claim 31, recipient of the flap is a non-human mammal.
